(19) United States
(12) Patent Application Publication
(10) Pub. No.: US 2014/0046177 A1
(43) Pub. Date: Feb. 13, 2014

(54) X-RAY RADIOGRAPHIC APPARATUS

(71) Applicants: Koichi Shibata, Kyoto-shi (JP); Yukio Mishina, Kyoto-shi (JP); Kazuhiro Mori, Kyoto-shi (JP)

(72) Inventors: Koichi Shibata, Kyoto-shi (JP); Yukio Mishina, Kyoto-shi (JP); Kazuhiro Mori, Kyoto-shi (JP)

(73) Assignee: SHIMADZU CORPORATION, Kyoto-shi, Kyoto (JP)

(21) Appl. No.: 13/988,059

(22) PCT Filed: Nov. 18, 2010
(86) PCT No.: PCT/JP2010/070528

§ 371 Date: Aug. 2, 2013

(51) Int. Cl. A61B 6/12

(52) U.S. Cl. CPC A61B 6/12
USPC 600/424

(57) ABSTRACT

Provided is an X-ray radiographic apparatus capable of accurately recognizing a position and orientation of a tip portion of an endoscope, wherein the X-ray radiographic apparatus includes a navigation processing part 70 for specifying a direction and position of an endoscope 50 and assisting an operation of the endoscope 50. This navigation processing part 70 includes: a position/direction detecting part 71 for detecting a position and direction of the tip portion of the endoscope 50; a CT image processing part 72 for processing a cone-beam CT image; and a virtual endoscopic image processing part 73 for processing a virtual endoscopic image. A coronal image, a sagittal image, an axial image, a front side radioscopic image, a lateral directional radioscopic image and a virtual endoscopic image obtained by cone-beam X-ray CT radiography are displayed in division on a monitor screen of a first monitor 30, and a real endoscopic image is displayed on a monitor screen of a second monitor 40.

### TECHNICAL FIELD

The present invention relates to an X-ray radiographic apparatus for use in endoscopy.

### BACKGROUND ART

For example, in the case where a bronchus is examined with an endoscope, it becomes important to accurately grasp a position of a tip portion of the endoscope. Conventionally, in order to specify a position of a tip portion of an endoscope, a three-dimensional image has been created as a virtual endoscopic image from multiple two-dimensional images obtained by X-ray CT photographing, and a position at which this virtual endoscopic image and an endoscopic image (real image) actually photographed with an endoscope are similar is detected as a current position of the tip portion of the endoscope (see Patent Literature 1).

Further, in this Patent Literature 1, it is described that the position of the tip portion of the endoscope may be detected by applying a magnetic field in a specific direction from an outside to a magnetic field sensor provided on a tip of the endoscope.

### CITATION LIST

### Patent Literature

Patent Literature 1: JP-A2009-56239

### SUMMARY OF INVENTION

### Technical Problem

Even though a configuration for specifying a position at which an image of a virtual endoscopic and an endoscopic image actually photographed by an endoscope are similar, as a current position of the endoscope as described above, since tissues of a human body has flexibility, it may likely be difficult to specify an accurate position of the tip portion of the endoscope or there may be a case where a position having similarity is not found and the position of the tip portion of the endoscope cannot be found.

Further, in the case of using a magnetic field sensor, although the position per se of the tip portion of the endoscope can be recognized, it is impossible to recognize an orientation of the endoscope, i.e., an insertion direction of the endoscope.

The present invention has been made in order to solve the above problems and an object thereof is to provide an X-ray radiographic apparatus capable of accurately recognizing a position and orientation of a tip portion of an endoscope.

### Solution to Problem

The invention described in claim 1 is an X-ray radiographic apparatus for use in endoscopy using an endoscope, characterized by including: a first photographing mechanism including: a first X-ray irradiation part and a first X-ray detector which are arranged to face each other across a subject; and an arm for rotatably supporting the first X-ray irradiation part and the first X-ray detector about a body axis of the subject, thereby performing a cone-beam X-ray CT photographing in a state of rotating the arm about the body axis of the subject and performing a radiography to the subject from a first direction in a state of stopping the arm; a second photographing mechanism including a second X-ray irradiation part and a second X-ray detector, thereby performing a radiography to the subject from a second direction perpendicular to the first direction; a display part capable of displaying a cone-beam X-ray CT photographic image photographed by the first photographing mechanism, a radioscopic image from the first direction photographed by the first photographing mechanism, and a radioscopic image from the second direction photographed by the second photographing mechanism; a position detecting part for detecting a three-dimensional coordinate position of a tip portion of the endoscope based on the radioscopic images from the first and second directions obtained by the first and second photographing mechanisms while performing the radiographies by the first and second photographing mechanisms, and a CT image processing part for superimposing the cone-beam X-ray CT photographic image at the position of the tip portion of the endoscope detected by the position detecting part with an image of the tip portion of the endoscope to thereby display a superimposed image on the display part.

The invention described in claim 2 is characterized in the invention described in claim 1 that the CT image processing part superimposes a coronal image, a sagittal image and an axial image obtained by the cone-beam X-ray CT photographing with the image of the tip portion of the endoscope to thereby display the superimposed image on the display part.

The invention described in claim 3 is characterized in the invention described in claim 2 by further including a virtual endoscope processing part for creating a three-dimensional image from multiple two-dimensional tomographic images obtained by the cone-beam X-ray CT photographing and displaying a three-dimensional image of the tip portion of the endoscope as a virtual endoscopic image on the display part.

The invention described in claim 4 is characterized in the invention described in claim 3 that the display part displays: the coronal image, sagittal image and axial image obtained by the cone-beam X-ray CT photographing; and the radioscopic image from the first direction photographed by the first photographing mechanism, the radioscopic image from the second direction photographed by the second photographing mechanism; and the virtual endoscopic image, on a single monitor screen in division, and characterized by further including a second display part for displaying an endoscopic image photographed by the endoscope.

The invention described in claim 5 is characterized in the invention described in claim 3 that the first X-ray irradiation part and the first X-ray detector are disposed at both ends of a C-type arm, the second X-ray detector is disposed at a central portion of the C-type arm, and further the second X-ray irradiation part is disposed at a position to face the second detector.

### Advantageous Effects of Invention

According to the invention described in claim 1, since the three-dimensional coordinate position of the tip portion of the endoscope is detected based on the radioscopic images obtained by the first and second photographing mechanisms while performing the radiographies and the cone-beam X-ray CT photographic image at the position of the tip portion of this endoscope is superimposed with the image of the tip portion of the endoscope to be thereby displayed on the display part, it becomes therefore possible to accurately and easily recognize the position and orientation of the tip portion of the endoscope.

According to the invention described in claim 2, since the coronal image, sagittal image and axial image obtained by the cone-beam X-ray CT photographing is superimposed with the image of the tip portion of the endoscope to be thereby displayed on the display part, it becomes therefore possible to easily recognize the position and orientation of the tip portion of the endoscope.

According to the invention described in claim 3, since the three-dimensional image is created from multiple two-dimensional tomographic images obtained by the cone-beam X-ray CT photographing and the three-dimensional image of the tip portion of the endoscope is displayed as the virtual endoscopic image on the display part, by comparing this virtual endoscopic image with an endoscopic image actually photographed by the endoscope, it becomes possible to easily recognize whether or not the recognized position and orientation of the tip portion of the endoscope are correct.

According to the invention described in claim 4, by displaying multiple pieces of image information on a single monitor screen in division, it is possible to easily recognize, and by displaying the endoscopic image photographed by the actual endoscope on the second display part, it becomes possible to accurately perform the endoscopy.

According to the invention described in claim 5, it becomes possible to perform the cone-beam X-ray CT photographing while moving the C-type arm and to perform the bidirectional radiography while fixing the C-type arm.

### BRIEF DESCRIPTION OF DRAWINGS

FIG. 1 is a schematic diagram of an X-ray radiographic apparatus according to the present invention.

FIG. 2 is a schematic diagram of an X-ray radiographic apparatus according to the present invention.

FIG. 3 is a schematic diagram showing a first monitor 30 and second monitor 40.

FIG. 4 is a schematic diagram of an endoscope 50 for use in endoscopy utilizing this X-ray radiographic apparatus.

FIG. 5 is a block diagram showing a main electrical configuration of the X-ray radiographic apparatus according to the present invention.

FIG. 6 is a schematic diagram showing a radioscopic image by a bidirectional radiography.

FIG. 7 is schematic diagram showing a cone-beam X-ray CT photographic image.

### DESCRIPTION OF EMBODIMENTS

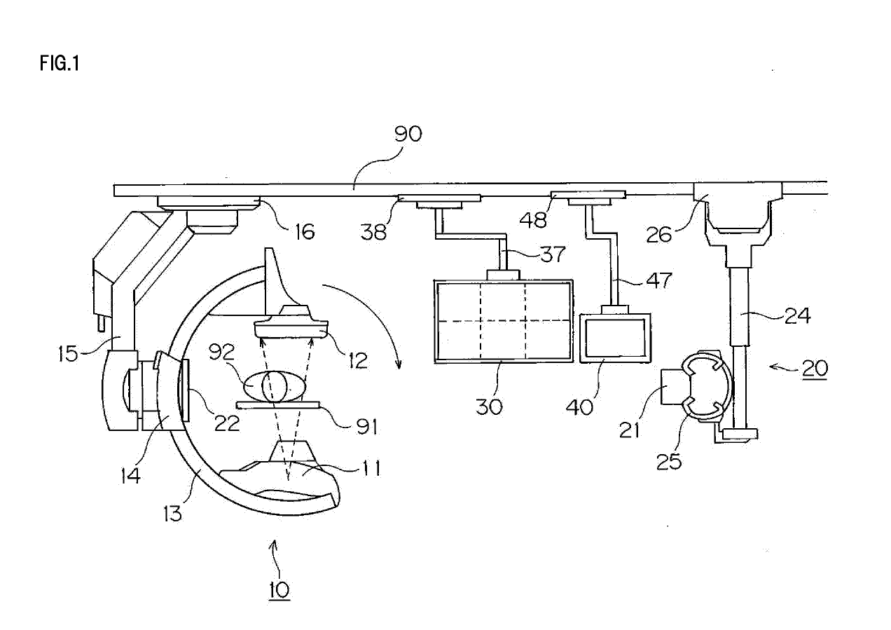

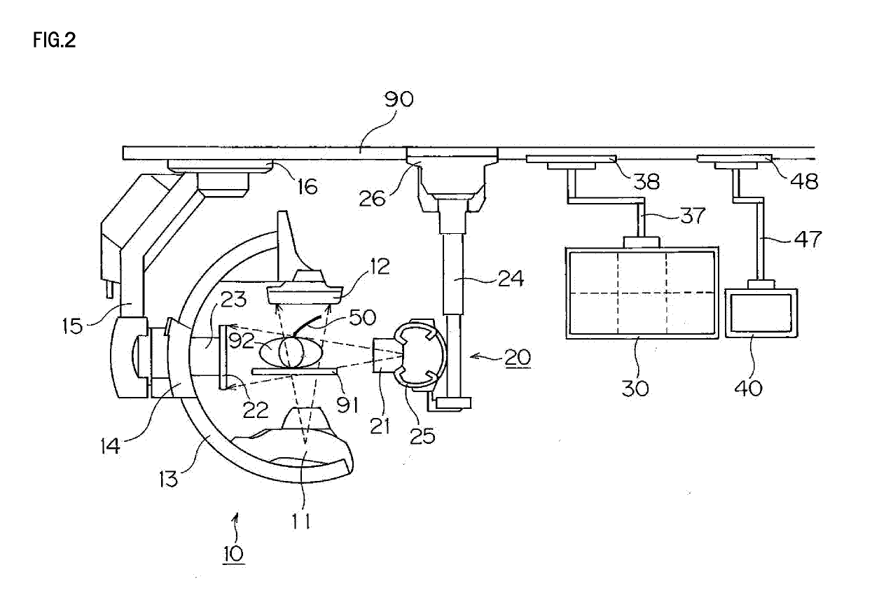

An embodiment of the present invention is described below with reference to the drawings. FIGS. 1 and 2 are schematic diagrams of an X-ray radiographic apparatus according to the present invention. In addition, FIG. 1 shows a state of performing a cone-beam X-ray CT photographing using a first photographing mechanism 10 and FIG. 2 shows a state of performing a bidirectional radiography using the first photographing mechanism 10 and a second photographing mechanism 20.

This X-ray radiographic apparatus includes: the first photographing mechanism 10 provided with an X-ray tube 11 as a first X-ray irradiation part and a flat panel detector (FPD) 12 as a first X-ray detector; the second photographing mechanism 20 provided with an X-ray tube 21 as a second X-ray irradiation part and a flat panel detector 22 as a second X-ray detector; a first monitor 30 as a display part; and a second monitor 40 as a second display part.

The first photographing mechanism 10 is provided with: a C-type arm 13 supporting the X-ray tube 11 and flat panel detector 12; and a supporting part 14 for slidably supporting this arm 13. This supporting part 14 is secured to a rail 90, which is fixed to a ceiling, via a base portion 16 and a hanging part 15. The arm 13 is rotatable about an axis line oriented in a vertical direction at the base portion 16 and is rotatable about an axis line oriented in a horizontal direction at the supporting part 14.

The arm 13 is formed with an arc-shaped guide portion (not shown) and the supporting part 14 is engaged with this guide portion to thereby slidably support the arm 13. And the arm 13 supports the X-ray tube 11 and flat panel detector 12 in a state that an axis line of X-rays from the X-ray tube 11 to the flat panel detector 12 coincides with a diameter of the circular arc forming the arm 13.

The second photographing mechanism 20 is provided with: a hanging part 24 for movably supporting the X-ray tube 21 up and down; a base portion 26 slidably supporting this hanging part 24 with respect to the rail 90 fixed to the ceiling; and a handle 25 for adjusting a position and direction of the X-ray tube 21. Further, the flat panel detector 22 in the second photographing mechanism 20 is supported in a state of being movable in a horizontal direction by an action of a sliding part 23 (see FIG. 2) with respect to the supporting part 14 in the first photographing mechanism 10.

The first monitor 30 is supported by a hanging part 37 and a base portion 38 which slidably supports this hanging part 37 with respect to the rail 90 fixed to the ceiling. Further, the second monitor 40 is supported by a hanging part 47 and a base portion 48 which slidably supports this hanging part 47 with respect to the rail 90 fixed to the ceiling.

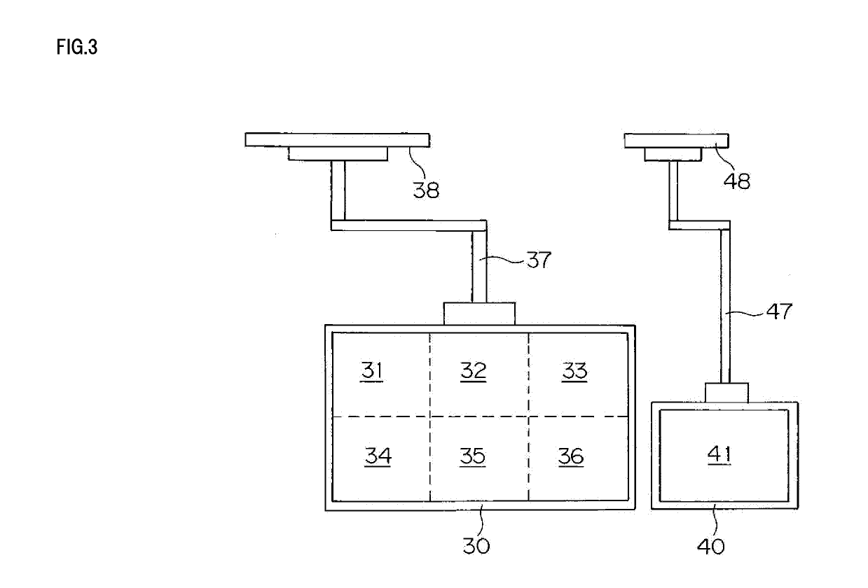

FIG. 3 is a schematic diagram showing the first monitor 30 and second monitor 40.

The first monitor 30 is intended to display in division various kinds of images to be described later on a single monitor screen, which includes six display regions 31, 32, 33, 34, 35 and 36 in the present embodiment. Further, the second monitor 40 is provided with a single monitor screen 41 for displaying an endoscopic image photographed by an endoscope 50 to be described later.

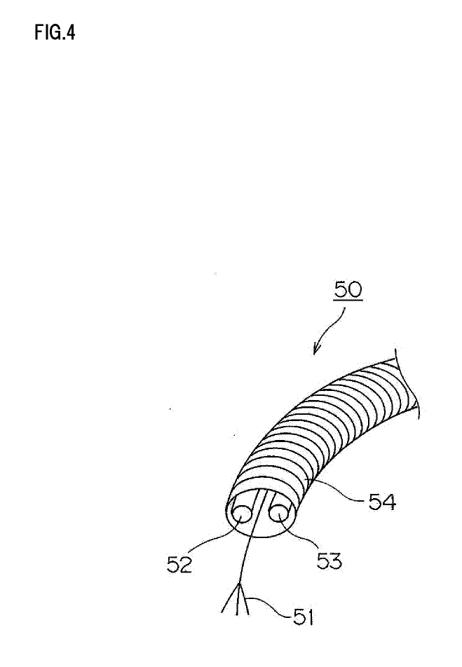

FIG. 4 is a schematic diagram of an endoscope 50 for use in the endoscopy utilizing this X-ray radiographic apparatus.

This endoscope 50 includes: forceps 51, a first optical fiber 52 connected to a camera, a second optical fiber 53 connected to a light source, and a cover 54.

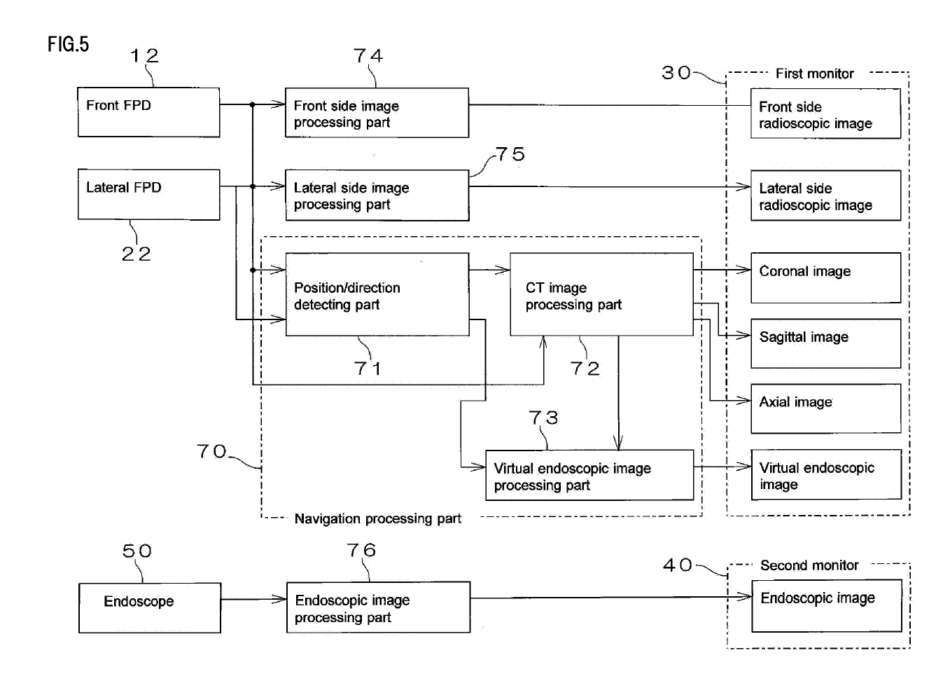

FIG. 5 is a block diagram showing a main electrical configuration of the X-ray radiographic apparatus according to the present invention.

The flat panel detector 12 in the first photographing mechanism 10 mentioned above is used in photographing of a frontal side (front side) at a time of radiography. An image signal from this flat panel detector 12 is image-processed by an image processing part 74 in the frontal side and a radioscopic image in the frontal side is displayed in the display region 34 in the first monitor 30. Further, the flat panel detector 22 in the second photographing mechanism 20 mentioned above is used for photographing in a lateral side (side surface side) at a time of radiography. An image signal from this flat panel detector 22 is image-processed by an image processing part 75 in the lateral side and a radioscopic image in the lateral side is displayed in the display region 35 in the first monitor 30.

This X-ray radiographic apparatus includes a navigation processing part 70 for specifying a direction and position of the endoscope 50 to assist an operation of the endoscope 50. This navigation processing part 70 includes a position/direction detecting part 71 for detecting a position and direction of the tip portion of the endoscope 50, a CT image processing part 72 for processing a CT image by the cone-beam X-ray CT photographing, and a virtual endoscopic image processing part 73 for processing a virtual endoscopic image.

Each of the image signals of the flat panel detector 12 and flat panel detector 22 at the time of radiography is sent to the position/direction detecting part 71. While performing the radiographies by both of the first photographing mechanism 10 and the second photographing mechanism 20, the position/direction detecting part 71 detects a three-dimensional coordinate position of the tip portion of the endoscope 50 and a direction of the endoscope 50 using an image process and the like based on the radiographic image obtained by the flat panel detector 12 and flat panel detector 22.

Further, the multiple two-dimensional tomographic images obtained by the flat panel detector 12 during the cone-beam X-ray CT photographing are sent to the CT image processing part 72. Moreover, information of the three-dimensional coordinate position and the direction thereof of the tip portion of the endoscope 50 detected by the position/direction detecting part 71 is also sent to this CT image processing part 72. Then, in the CT image processing part 72, the cone-beam X-ray CT photographic image in the position of the tip portion of the endoscope 50 detected by the position/direction detecting part 71 is superimposed with the image of the tip of the endoscope 50 to be displayed in the first monitor. More specifically, the CT image processing part 72 renders: a coronal image obtained by the cone-beam X-ray CT photographing; a sagittal image obtained by the cone-beam X-ray CT photographing; and an axial image obtained by the cone-beam X-ray CT photographing, to be displayed, respectively, in the display region 31 in the first monitor 30, in the display region 32 in the first monitor 30 and in the display region 33 in the first monitor 30, while being superimposed with the image of the tip of the endoscope 50.

Further, the information of the three-dimensional coordinate position and the direction thereof of the tip portion of the endoscope 50 detected by the position/direction detecting part 71 is sent to the virtual endoscopic image processing part 73. Also, the multiple two-dimensional tomographic images obtained by the flat panel detector 12 at the time of the cone-beam X-ray CT photographing are sent from the CT image processing part 72 to the virtual endoscopic image processing part 73. Then, the virtual endoscopic image processing part 73 creates a three-dimensional image from the multiple two-dimensional tomographic images obtained by the cone-beam X-ray CT photographing and displays the three-dimensional image of the tip portion of the endoscope 10 as the virtual endoscopic image in the display region 36 in the first monitor 30. At this time, the three-dimensional coordinate position of the tip portion of the endoscope 50 and the direction of the endoscope 50 detected by the position/direction detecting part 71 are used.

Further, the endoscopic image photographed by the endoscope 50 is image-processed by the endoscopic image processing part 76 and displayed as a real endoscopic image in the display region 41 of the second monitor 40.

When performing the endoscopy using the X-ray radiographic apparatus having a configuration as described above, the cone-beam X-ray CT photographing is first performed using the first photographing mechanism 10. At this time, as shown in FIG. 1, the X-ray tube 21 in the second photographing mechanism 20 is moved to a retracted position spaced from the first photographing mechanism 10 along the rail 90. Further, the flat panel detector 22 is moved to a retracted position spaced from the subject 92 by an action of the sliding part 23 shown in FIG. 2. In this state, the arm 13 is rotated about a body axis of the subject 92 in a state that the subject 92 is rendered to lie on its back on a table 91 to thereby perform the cone-beam X-ray CT photographing. The multiple two-dimensional tomographic images obtained by this are sent to the navigation processing part 70.

Upon completion of the above preparing process, the endoscopy is started using the endoscope 50. At this time, the bidirectional radiography is performed. That is, as shown in FIG. 2, the X-ray tube 21 in the second photographing mechanism 20 is moved to a photographing position close to the subject 92 along the rail 90. Also, the flat panel detector 22 is moved to a photographing position close to the subject 92 by an action of the sliding part 23. Then, the bidirectional radiography is performed using the first photographing mechanism 10 and second photographing mechanism 20.

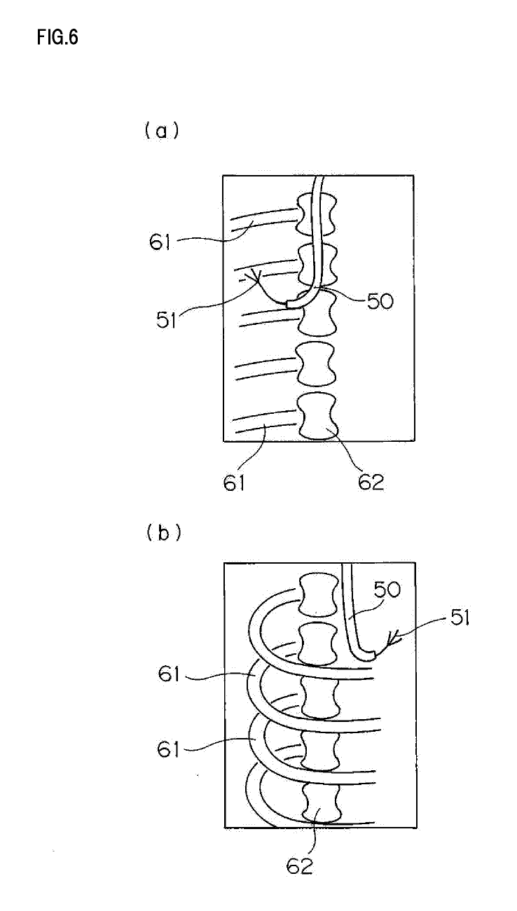

FIG. 6 is a schematic diagram showing a radioscopic image by the bidirectional radiography.

FIG. 6(a) shows the radioscopic image in the lateral direction photographed by the second photographing mechanism 20. This radioscopic image is displayed in the display region 35 in the first monitor 30. FIG. 6(b) shows the radioscopic image in the frontal direction photographed by the first photographing mechanism 10. This radioscopic image is displayed in the display region 34 in the first monitor 30. Forceps 51 of the tip portion of the endoscope 50 are displayed in these radioscopic images. It is noted that reference numeral 61 denotes a rib and reference numeral 62 denotes a spine (backbone) in this drawing.

Further, at the same time of this, while each of the coronal image, sagittal image and axial image obtained by the cone-beam X-ray CT photographing is superimposed with the image of the tip of the endoscope 50 and displayed in the first monitor 30 by the navigation processing part 70, the three-dimensional image of the tip portion of the endoscope 10 obtained from the multiple two-dimensional tomographic images by the cone-beam X-ray CT photographing is displayed as the virtual endoscopic image in the first monitor 30.

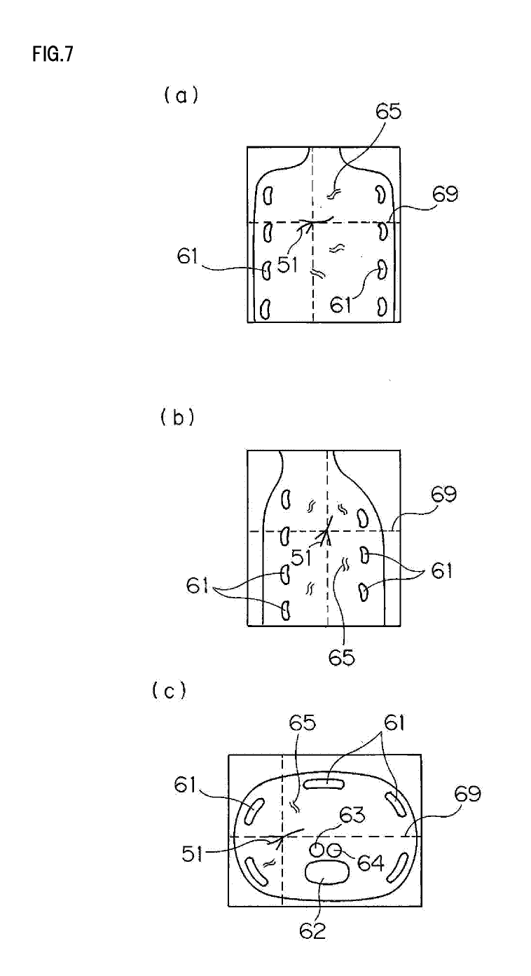

FIG. 7 is a schematic diagram showing the cone-beam X-ray CT photographic image.

FIG. 7(a) shows the coronal image by the cone-beam X-ray CT photographing. This coronal image is displayed in the display region 31 in the first monitor 30. FIG. 7(b) shows the sagittal image by the cone-beam X-ray CT photographing. This sagittal image is displayed in the display region 32 in the first monitor 30. FIG. 7(c) shows the axial image by the cone-beam X-ray CT photographing. This axial image is displayed in the display region 33 in the first monitor 30. It is noted that, in this drawing, reference numeral 61 denotes a rib, reference numeral 62 a spine (backbone), reference numeral 63 a vena cava, reference numeral 64 an aorta and reference numeral 65 the other vessels, respectively. Further, at a position of the forceps 51 of the tip portion of the endoscope 50 in each of these display images, a cross-shaped mark line 69 is displayed. These displayed images are varied with movement of the endoscope 50.

Thus, by superimposing each of the coronal image, sagittal image and axial image obtained by the cone-beam X-ray CT photographing with the image of the tip of the endoscope 50 to be displayed, it becomes possible to recognize the position and orientation of the tip portion of the endoscope more easily. Therefore, it becomes possible to improve operability by performing navigation of the operation of the endoscope in the endoscopy.

Further, at this time, as described above, the three-dimensional image of the tip portion of the endoscope 10 obtained from the multiple two-dimensional tomographic images by the cone-beam X-ray CT photographing is displayed as the virtual endoscopic image in the display region 36 of the first monitor 30. Further, a real endoscopic image photographed by the endoscope 50 is displayed as a real endoscopic image in the display region 41 of the second monitor 40 disposed closely to the display region 36 of the first monitor 30. These virtual endoscopic image and real endoscopic image are varied with movement of the endoscope.

Then, in the case where these virtual endoscopic image and real endoscopic image are substantially identical, it is possible to confirm that the previously recognized position and orientation of the tip portion of the endoscope are accurate, and in the case where these virtual endoscopic image and real endoscopic image are significantly different, it becomes possible to confirm that the previously recognized position and orientation of the tip portion of the endoscope are erroneous.

It is noted that, in the embodiment described above, each of the coronal image, sagittal image and axial image obtained by the cone-beam X-ray CT photographing with the image of the tip of the endoscope 50 to be displayed. However, a frontal image and lateral image may be displayed, and 3D display may be performed as the three-dimensional information.

Further, in the embodiment described above, although the coronal image, sagittal image and axial image obtained by the cone-beam X-ray CT photographing, and the frontal side radioscopic image photographed by the first photographing mechanism 10, the radioscopic image from the lateral direction photographed by the second photographing mechanism 20 and virtual endoscopic image are displayed in division on the monitor screen of the first monitor 30 while the real endoscopic image is displayed on the monitor screen of the second monitor 40, these may be displayed on different monitor screens, or these may be switched to be displayed.

### REFERENCE SIGNS LIST

10 . . . First photographing mechanism
11 . . . X-ray tube
12 . . . Flat panel detector
13 . . . Arm
14 . . . Supporting part
15 . . . Hanging part
20 . . . Second photographing mechanism
21 . . . X-ray tube
22 . . . Flat panel detector
24 . . . Hanging part
30 . . . First monitor
37 . . . Hanging part
40 . . . Second monitor
47 . . . Hanging part
50 . . . Endoscope
51 . . . Forceps
70 . . . Navigation processing part
71 . . . Position/direction detecting part
72 . . . CT image processing part
73 . . . Virtual endoscopic image processing part
74 . . . Image processing part
75 . . . Image processing part
76 . . . Endoscopic image processing part
90 . . . Rail
91 . . . Table
92 . . . Subject

1. An X-ray radiographic apparatus for use in endoscopy using an endoscope, characterized by comprising:
a first photographing mechanism including: a first X-ray irradiation part and a first X-ray detector which are arranged to face each other across a subject; and an arm for rotatably supporting the first X-ray irradiation part and the first X-ray detector about a body axis of the subject, thereby performing a cone-beam X-ray CT photographing in a state of rotating the arm about the body axis of the subject and performing a radiography to the subject from a first direction in a state of stopping the arm;
a second photographing mechanism including a second X-ray irradiation part and a second X-ray detector, thereby performing a radiography to the subject from a second direction perpendicular to the first direction;
a display part capable of displaying a cone-beam X-ray CT photographic image photographed by the first photographing mechanism, a radioscopic image from the first direction photographed by the first photographing mechanism, and a radioscopic image from the second direction photographed by the second photographing mechanism;
a position detecting part for detecting a three-dimensional coordinate position of a tip portion of the endoscope based on the radioscopic images from the first and second directions obtained by the first and second photographing mechanisms while performing the radiographies by the first and second photographing mechanisms, and
a CT image processing part for superimposing the cone-beam X-ray CT photographic image at the position of the tip portion of the endoscope detected by the position detecting part with an image of the tip portion of the endoscope to thereby display a superimposed image on the display part.

2. The X-ray radiographic apparatus according to claim 1, wherein the CT image processing part superimposes a coronal image, a sagittal image and an axial image obtained by the cone-beam X-ray CT photographing with the image of the tip portion of the endoscope to thereby display the superimposed image on the display part.

3. The X-ray radiographic apparatus according to claim 2 further comprising a virtual endoscope processing part for creating a three-dimensional image from multiple tomographic two-dimensional images obtained by the cone-beam X-ray CT photographing and displaying a three-dimensional image of the tip portion of the endoscope as a virtual endoscopic image on the display part.

4. The X-ray radiographic apparatus according to claim 3, wherein the display part displays: the coronal image, sagittal image and axial image obtained by the cone-beam X-ray CT photographing; and the radioscopic image from the first direction photographed by the first photographing mechanism, the radioscopic image from the second direction photographed by the second photographing mechanism; and the virtual endoscopic image, on a single monitor screen in division, and
further comprising a second display part for displaying an endoscopic image photographed by the endoscope.

5. The X-ray radiographic apparatus according to claim 3, wherein the first X-ray irradiation part and the first X-ray detector are disposed at both ends of a C-type arm, the second X-ray detector is disposed at a central portion of the C-type arm, and further the second X-ray irradiation part is disposed at a position to face the second detector.
